(19) United States
(12) Patent Application Publication
(10) Pub. No.: US 2007/0098695 A1
(43) Pub. Date: May 3, 2007

(54) Methods of islet separation during isolation

(76) Inventors: James DeOlden, Purcellville, VA (US); Jonathan Lakey, Edmonton (CA)

Correspondence Address: Eric A. Lerner;Eckert Seamans Cherin & Mellott, LLC, 44th Floor, 600 Grant Street, Pittsburgh, PA, 15219, US

(21) Appl. No.: 11/263,463

(22) Filed: Oct. 31, 2005

(51) Int. Cl. A61K 35/39; C12N 5/08

(52) U.S. Cl. 424/93.7; 435/366

(57) ABSTRACT

The present invention relates to methods for improving the viability and recovery of islets that are separated from a donor organ for subsequent transplantation. In a preferred embodiment, the islets are separated from a donor pancreas and transplanted into the liver of a diabetic patient. A protective agent is mixed with a separating solution to form a protective separating solution which is utilized to separate the islets cells from other cells after the donor pancreas is digested with an enzyme. The protective agent increases the viscosity of the solution during the separation process, creating a cushioning effect of islets, thereby enhancing their viability and health so that the islets' membranes may withstand a vigorous isolation procedure such as the Edmonton Protocol.

### FIELD OF THE INVENTION

The present invention relates to methods of isolating and transplanting islets, and more particularly relates to the use of a protective agent during one or a multiplicity of islet separating steps during islet isolation to enhance the viability of the islets by reducing physical stress on the islets and increasing the amount of islets that can be successfully transplanted.

### BACKGROUND INFORMATION

An islet is a multi-cellular entity that produces insulin within the pancreas, wherein each islet is typically about 100 to 600 microns in diameter and contains greater than 1000 cells. The average person has about a million islets, comprising approximately three percent of the total mass of cells in the pancreas. The pancreas contains the islets of Langerhans, which house beta cells that produce insulin or other hormones. The beta cells monitor glucose levels in the blood and release finely measured amounts of insulin to counterbalance glucose peaks. Type I and II diabetes develop when more than 90 percent of these beta cells are damaged and destroyed.

An islet, like an organelle, has a distinctive shape and function, and contains more than one type of cell (e.g., the beta cell) within the islet unit. A loosely defined membrane surrounds each islet. If the membrane surrounding the islet breaks, the overall islet will become dysfunctional and the cells inside the islets will fall apart. Islet membrane breakage may readily occur under certain circumstances, as islet membranes are typically fragile and may break when placed under undue stress. One such circumstance of undue stress is created when conveying a solution past an islet in a generally perpendicular path with respect to the islet's membrane. The viscosity of the solution as it passes the islets creates a condition favorable to cellular membrane damage.

A therapy well known in the art called the Edmonton Protocol, as well as other evolving therapies, transplant healthy human islets into type 1 recipients. Islet transplantation using the Edmonton Protocol is described in Shapiro, Ryan, and Lakey, Clinical Islet Transplantation—State of the Art, Transplantation Proceedings, 33, pp. 3502-3503 (2001); Ryan et al., Clinical Outcomes and Insulin Secretion After Islet Transplantation With the Edmonton Protocol, Diabetes, Vol. 50, April 2001, pp. 710-719; Ryan et al., Continued Insulin Reserve Provides Long-Term Glycemic Control, Diabetes, Vol. 51, July 2002, pp. 2148-2157; and, the New England Journal of Medicine (2000). Once in the liver, the islets begin producing and secreting insulin. The Edmonton Protocol includes 7-10 defined steps depending on the isolation method employed. The first step involves the delivery of an enzyme to a donor pancreas via the pancreatic duct, which digests the pancreas tissue, but does not digest the islets. Following the digestion step, there are several successive steps for separating the islets from other cells in the pancreas, called islet separation, including the use of a separating solution that is conveyed generally perpendicular to the islets. The separated islets are purified and are transplanted into the main vessel of the liver, known as the portal vein. The liver is able to regenerate itself when damaged, building new blood vessels and supporting tissue. Therefore, when islets are transplanted into the liver, it is believed that new blood vessels form to support the islets. The insulin that the cells produce is absorbed into the blood stream through these surrounding vessels and distributed through the body to control glucose levels in the blood.

Altogether, the steps of the Edmonton Protocol create a vigorous process that compromises the viability of islets, which have a fragile, three-dimensional structure and require large amounts of oxygen for viability and materials that can support the fragile membrane during the vigorous isolation process. During the process, islets may be damaged or destroyed due to non-optimal conditions of oxygen delivery and the physical stress of shear which damages the outer cell membrane during the procedure, affecting the yield of healthy islets that are retrieved from a given donor pancreas. Furthermore, islet transplantation is severely limited by donor availability; frequently, two pancreata are required to obtain insulin independence in one patient. As a result, there is a need for improved methods of isolation and transplantation that mitigate damage to islets and permit insulin independence from a single donor.

Improvements in the rate of single donor transplantation have been reported using the two layer method (TLM) of pancreas preservation; see, e.g., Salehi et al., Ameliorating ischemic injury during preservation and isolation of human islet cells using the two layer method with perfluorocarbon and University of Wisconsin solution, Transplantation 2005 (in press); Lakey et al., Human Pancreas Preservation Prior to Islet Isolation, Cell Preservation Technology, Vol. 1, No. 1, 2002, pp. 81-87; and Tsujimura et al., Human Islet Transplantation From Pancreases with Prolonged Cold Ischema Using Additional Preservation by the Two-Layer (UW Solution/Perfluorochemical) Cold-Storage Method, Transplantation, Vol. 74, No. 12, Dec. 27, 2002, pp. 1687-1691. TLM involves the use of University of Wisconsin (UW) solution along with a perfluorocarbon (PFC) such as perfluorodecalin to preserve a human pancreas. UW has been employed in organ preservation for many years. It contains cell impermeant agents such as lactobionic acid that prevents cell swelling during cold storage, as well as glutathione, which works as an antioxidant, and adenosine, important for adenosine triphosphate synthesis. PFC, which is immiscible in water, has been helpful in pancreas preservation because of its high oxygen storage capability and low oxygen-binding constant, which allow it to store large amounts of oxygen for effective delivery to the ischemic organ. According to TLM methodology, the organ is preserved by immersing it in a container of the UW and PFC, where the organ is positioned to sit at the interface of the two liquids.

There remains a need, however, to develop an isolation process that improves the viability of the islets during the vigorous processes of separation of the islets from other cells in donor organs such as pancreata by reducing stress inflicted on the islets' fragile membranes, increasing the number of viable islets available for transplantation into a destination organ such as a liver.

### SUMMARY OF THE INVENTION

The present invention provides methods for improving the viability and recovery of islets that are separated from a donor organ for subsequent transplantation. In a preferred embodiment, the islets are separated from a donor pancreas and transplanted into the liver of a diabetic patient. The present invention includes the addition of a protective agent, preferably dextran, a long chain polymer of glucose (i.e., (C₆H10O₅)ₙ) having variable molecular weight, to a separating solution for separating the islets to be transplanted. More preferably, the present invention includes the addition of a protective agent that is clinical grade dextran, which has a molecular weight of approximately 60,000 Daltons.

The invention provides a method including an addition of a protective agent to a separating solution to form a protective separating solution. The protective separating solution is utilized in the Edmonton Protocol after the digestion stage of the Edmonton Protocol, wherein the pancreas tissue is digested. The protective agent may be subsequently added during any of several successive separation steps that separate the islets from other cells in the pancreas and prior to a final purification step wherein a density gradient centrifugation is utilized. The protective agent reduces the amount of stress incurred by the fragile islet membranes by reducing shear imposed on the islets by the separating solution. By doing so, the present invention rescues islets that would otherwise be damaged or destroyed during the vigorous separation procedures. The separated islets may thereafter be injected into the portal vein of a liver where it is believed they develop a blood supply and assist in producing insulin and regulating blood glucose levels.

An object of the present invention is to provide a method of isolating islets comprising introducing a protective agent to a donor organ during separation of the islets from the donor organ.

Another object of the present invention is to provide a method of transplanting islets comprising introducing a protective agent to a donor organ during separation of the islets from other cells in the donor organ, and transplanting separated islets into a destination organ.

Another object of the present invention is to provide a method of transplanting islets comprising introducing dextran to a donor organ during separation of the islets from the donor organ, and transplanting separated islets into a destination organ.

Another object of the present invention is to increase the viscosity of solution moving past the islets during the separation process thereby creating a cushioning effect.

Another object of the present invention is to decrease the level of shear damage imposed on the islets during the separation process.

Another object of the present invention is to rescue the donor organs that would otherwise be considered unsuitable for use.

Another object of the present invention is to decrease the number of islets that are damaged or destroyed during the isolation and transplantation process and increase the yield of viable, healthy, transplantable cells.

Another object of the present invention is to mitigate the need for multiple donor organs to achieve insulin independence.

Another object of the present invention is to allow donor organs to withstand a longer transit time.

Another object of the present invention is to standardize isolation procedures that are used for donor organs of varying quality.

These and other aspects of the present invention will become more readily apparent from the following detailed description and appended claims.

### DETAILED DESCRIPTION

The present invention provides methods for improving the viability and recovery of islets that are separated from a donor organ for subsequent transplantation. In a preferred embodiment, the islets are separated from a donor pancreas and transplanted into the liver of a diabetic patient. While the description contained herein primarily refers to cell transplantations into livers, it is to be understood that the invention may be utilized for other transplant destinations, such as testes.

As used herein, the terms “patient”, “donor”, and “donee” refer to members of the animal kingdom, including humans.

As used herein, the term “protective agent” refers to an agent that, when added to a separating solution, reduces the stress and/or damage on islets during a separation step of an isolation procedure.

As used herein, the term islet “isolation” includes islet separation and any number of other steps.

The present invention provides an introduction of a protective agent into a donor pancreas during an islet separation process, preferably during use of the “Edmonton Protocol”. Specifically, introduction of the protective agent occurs after digestion of the donor pancreas with an enzyme and prior to purification of the islets with a density gradient centrifugation.

The introduction of the protective agent will occur during one or a multiplicity of separation steps wherein the islets are separated from other cells in the donor organ. The introduction of the protective agent can occur once or a multiplicity of times prior to islet purification. This introduction may be accomplished by adding the protective agent to a separating solution to form a protective separating solution prior to the introduction of the separation solution on the islets. The protective separating solution is then utilized during one or a multiplicity of separating steps. Alternatively, the protective agent may be introduced simultaneously on the islets along with the separating solution to form a protective separating solution therein. The separating solution is a solution known in the art for separating islet cells such as those referenced in the Edmonton Protocol.

The protective agent increases the viscosity of the separating solution providing a cushioning effect on the membrane against the stress caused by the solution. By reducing stresses on the islets' membranes, the protective agent enhances islet health and viability so they may withstand a vigorous separation/isolation procedure such as the Edmonton Protocol. As a result, the present invention rescues islets that would otherwise be damaged or destroyed by the isolation and transplantation procedure.

In preferred embodiments, the protective agent is dextran, a long chain polymer of (C₆H₁₀O₅)ₙ, that can occur in various molecular weights. In more preferred embodiments, the protective agent is clinical grade dextran, wherein the dextran has a molecular weight of approximately 60,000 Daltons.

The amount of protective agent added to the separating solution may vary within the spirit of the invention. The amount of protective agent employed and the total amount of separating solution used may vary considerably and/or depart from the values stated above depending on the quality, health, and size of the donor organ, the time at which it was removed from the donor, and the method of transporting the organ. Preferably, the amount of protective agent added is about 1 to 20 weight percent of the separating solution. The weight percent of protective agent added may be greater than 20 weight percent, but this may affect the separation ability of the separation solution and separation ability may decrease.

In alternate embodiments, other protective agents may be used. In one embodiment, albumin, a protein, is utilized as a protective agent. Albumin, like clinical grade dextran, has a molecular weight of approximately 60,000 Daltons. In alternate embodiments, protective agents that have molecular weights in ranges other than 60,000 Daltons are used. These protective agents include, but are not limited to, carboxyl methyl cellulose (CMC) and dextrans of molecular weights other than 60,000 Daltons.

The protective agent is preferably mixed into the separating solution and introduced into the digested donor organ to separate the islets from other cells and organelles in the digested donor organ. The mixture is introduced into the digested donor organ using instruments as disclosed by the Edmonton Protocol, for example, using a thin needle, canula, plastic tube, or similar device.

In preferred embodiments, the Edmonton Protocol is used to separate the islets from other cells in a digested donor organ, preferably a donor pancreas or pancreata. The Edmonton Protocol involves multiple steps, including distention of the pancreas through ductal perfusion, followed by enzymatic and mechanical digestion, and purification of islets using density gradient centrifugation. The separation step is a vigorous process that typically damages or destroys many islets, leading to a low yield of viable, transplantable, post-isolation cells. However, with the addition of the protective agent, more islets survive a potential breakage of membranes, and therefore a greater number of these cells survive the process.

Once the islets are isolated, the islets are introduced into a destination organ. In one embodiment, the destination organ is a liver of a diabetic patient. However, in other embodiments, any other patient organ that can benefit from the method can be used. For example, other destination organs include, but are not limited to, a patient's scrotum, a patient's kidney or a vitreous of a patient's eye.

In alternative embodiments, other protocols alternative to the Edmonton Protocol incorporate the present invention, namely the addition of a protective agent to reduce the stresses on islets' membranes, provided the other protocols also separate islets from other cells of a donor organ.

### EXAMPLE 1

Human donor pancreases were obtained to test the effect of clinical grade dextran on cellular viability, as shown in Tables 1 and 2. As shown in Table 1, five experimental donor pancreases, referred to as donors 1, 2, 3, 4 and 5, utilized separation solution that included clinical grade dextran, wherein the weight percent of clinical grade dextran added was 3%, 3%, 5%, 5% and 5% respectively. For control, shown in Table 2, five donor pancreata, referenced as donors 6, 7, 8, 9 and 10, did not receive separation solution that included clinical grade dextran. Aside from this difference, all islets from the donors were otherwise isolated using the standard Edmonton Protocol.

TABLE 1

| | Dextran | | | | | |
|---|---|---|---|---|---|---|
| Donor # | wt % | Pre IE | Post IE | % Pre IE | Viability % | Tx |
| 1 | 3 | 745096 | 452360 | 0.61 | 83 | Yes |
| 2 | 3 | 215236 | 277268 | 1.29 | 93 | Yes |
| 3 | 5 | 413546 | 281757 | 0.68 | 92 | Yes |
| 4 | 5 | 340901 | 286866 | 0.84 | 89 | Yes |
| 5 | 5 | 1034556 | 429471 | 0.42 | 93 | Yes |
| Average | | 549867 | 345544 | 0.63 | 90 | 100% |

TABLE 2

| | Dextran | | | | | |
|---|---|---|---|---|---|---|
| Control # | wt % | Pre IE | Post IE | % Pre IE | Viability % | Tx |
| 6 | 0 | 425615 | 215413 | 0.51 | 52 | No |
| 7 | 0 | 632086 | 359492 | 0.57 | 95 | Yes |
| 8 | 0 | 57287 | 58906 | 1.03 | 78 | No |
| 9 | 0 | 410121 | 212164 | 0.52 | 83 | Yes |
| 10 | 0 | 365116 | 61196 | 0.17 | 85 | No |
| Average | | 378045 | 181434 | 0.48 | 79 | 40% |

The above tables compare islet equivalence (IE) of the donor pancreases prior to isolation of the islets by the Edmonton Protocol (Pre IE) and after isolation (Final IE). IE is a normalized unit that measures physical amounts of islets. Pre IE refers to the number of islet equivalence at the start of the process. Post IE refers to the number of islet equivalence at the end of the process. % Pre IE refers to the post IE divided by pre IE. Tx refers to whether the cells were transplanted.

As can be seen in the tables, a greater percentage of islets survived in the donors that were processed with clinical grade dextran. % Pre IE for donors 1, 2, 3, 4 and 5 were 60.7, 129, 68.1, 84.0, and 41.5 percent, respectively, for a total average of 57.6%. In contrast, the % Pre IE for the control donors 6, 7, 8, 9 and 10 were 50.6, 56.9, 103, 51.7, and 16.8 percent, respectively, for a total average of 48.0%. The difference of the average percent of the Final IE as a percentage of Pre IE of donors with dextran vs. control was a 20% increase for the donors with dextran.

Further, viability of the islet cells was measured. Viability was measured with a stain that colors viable islets and not non-viable islets. Any type of stains known in the art for this purpose are suitable. Islets were counted on a grid as is known in the art. As shown in Table 1, the viability of donors 1, 2, 3, 4 and 5 were 83, 93, 92, 89, and 93 percent, respectively, with a total average of 90% viability. Preferably, 300,000 viable islets cells are needed for transplant, but lower amounts can be transplanted if their ratings are good. As a result of the Percentage of viable islets and the Final IE islet count, 100% of the donors that utilized clinical grade dextran, produced islets viable for transfer into a patient.

Control donors 6, 7, 8, 9 and 10 had islet viability of 52, 95, 78, 83, and 85 percent, respectively, with a total average of 79.0% viability. Further, due to low numbers of viable islets, control donors 1, 3 and 5 were inadequate for transfer into a patient. Thus, in contrast to the 100% transfer rate of donors treated with clinical grade dextran, only 40% of control donors were viable for transfer.

### EXAMPLE 2

A human donor pancreas was obtained to test the effect of clinical grade dextran on cellular viability, as shown in Table 3. The donor pancreas was treated with a solution of 10 weight % clinical grade dextran during the separation steps of the Edmonton Protocol.

TABLE 3

| | Dextran | | | | | |
|---|---|---|---|---|---|---|
| Donor # | wt % | Pre IE | Post IE | % Pre IE | Viabiliy % | Tx |
| 11 | 10 | 632443 | 338225 | 0.53 | 90 | Yes |

As shown in the Table 3, Final IE was 54% of Pre IE. However, 89.6% of the islets were found to be viable, and the islets were found to be sufficient for transplant.

Whereas particular embodiments of this invention have been described above for purposes of illustration, it will be evident to those skilled in the art that numerous variations of the details of the present invention may be made without departing from the invention as defined in the appended claims.

What is claimed is:

1. A method of isolating islets comprising:
adding a protective agent to a separating solution to form a protective separating solution, and
using the protective separating solution to separate the islets from a donor organ during a separation step, wherein the protective separating solution reduces damage to the islets during the separation step.

2. The method of claim 1, wherein the donor organ comprises one or a multiplicity of human pancreases.

3. The method of claim 1, wherein the protective agent is dextran.

4. The method of claim 3, wherein the dextran is clinical grade dextran.

5. The method of claim 1, wherein the protective agent is selected from the group consisting of dextran, albumin, carboxyl methyl cellulose and a combinations thereof.

6. The method of claim 1, wherein the protective agent comprises about 1 to 20 percent by weight of the protective separating solution.

7. The method of claim 1, wherein the protective separating solution is introduced into the donor organ.

8. The method of claim 1, wherein the islets are separated from the donor organ according to the Edmonton Protocol.

9. The method of claim 1, wherein the protective agent rescues a donor organ that would otherwise be considered unsuitable for use.

10. A method of transplanting islets comprising:
adding a protective agent to a separating solution to form a protective separating solution,
using the protective separating solution to separate islets from a donor organ, and
transplanting the separated islets into a destination organ.

11. The method of claim 10, wherein the donor organ comprises one or a multiplicity of human pancreases.

12. The method of claim 10, wherein the destination organ is selected from the group consisting of a liver, kidney, scrotum and a vitreous of an eye.

13. The method of claim 10, wherein the protective agent is dextran.

14. The method of claim 13, wherein the dextran is clinical grade dextran.

15. The method of claim 10, wherein the protective agent is selected from the group consisting of dextran, albumin, carboxyl methyl cellulose and a combination thereof.

16. The method of claim 10, wherein the protective agent comprises about 1 to 20 percent by weight of the protective separating solution.

17. The method of claim 10, wherein the protective separating solution is introduced into the donor organ.

18. The method of claim 10, wherein the islets are separated from the donor organ according to the Edmonton Protocol.

19. The method of claim 10, wherein the protective agent rescues a donor organ that would otherwise be considered unsuitable for use.

20. The method of claim 10, wherein the protective agent reduces stress or damage on the islets when using the solution to separate islets from a donor organ.
